(19) United States
(12) Patent Application Publication
(10) Pub. No.: US 2025/0064082 A1
(43) Pub. Date: Feb. 27, 2025

(54) SOLID PLANT PROTEIN COMPOSITIONS WITH IMPROVED PHYSICAL AND/OR ORGANOLPETIC PROPERTIES AND METHODS OF MAKING AND USING THEREOF

(71) Applicant: Eat Just, Inc., Alameda, CA (US)

(72) Inventors: Michael Jamos, Alameda, CA (US); Vivek Sharma, Alameda, CA (US); Tyler Hurlburt, Alameda, CA (US); Ying Zhao, Alameda, CA (US)

(21) Appl. No.: 18/732,530

(22) Filed: Jun. 3, 2024

Related U.S. Application Data
(60) Provisional application No. 63/505,816, filed on Jun. 2, 2023.

(51) Int. Cl. A23J 3/14; A23J 1/14; A23J 3/22; C12N 9/10

(52) U.S. Cl. CPC A23J 3/14; A23J 1/148; A23J 3/227; C12N 9/1044; C12Y 203/02013

(57) ABSTRACT

Solid purified plant protein compositions and methods of making and using thereof are described. In some embodiments, the plant protein compositions exhibit one or more desired physical and/or functional properties, e.g., viscosity, gelation capability, gel quality, protein stability, and/or one or more desired organoleptic properties (e.g., aroma, taste, etc.). The solid plant protein composition can contain one or more plant proteins; one or more phosphate salts; one or more Group I and/or Group II metal halide salts; and optionally, one or more citrate salts. The method for producing a solid plant protein composition can include: suspending a purified plant protein in a solvent; adding a phosphate salt, a Group I and/or Group II metal halide salt, and optionally a citrate salt, to form a plant protein composition; and drying the plant protein composition.

### CROSS-REFERENCE TO RELATED APPLICATION

The present application claims priority to and benefit of U.S. Provisional Application No. 63/505,816 filed Jun. 2, 2023, the content of which is hereby incorporated by reference in its entirety.

### FIELD OF THE INVENTION

The present disclosure relates to solid plant protein compositions, methods of making the same, and food products containing the plant protein compositions.

### BACKGROUND

Use of plant-based proteins such as soy and pea as animal protein substitutes have garnered increasing attention largely as consumers seek alternatives to conventional animal-based products. However, replicating functional and/or organoleptic (sensory) properties of specific animal proteins (taste, aroma, texture, firmness, gelation properties, etc.) are still challenges that need to be addressed.

Conventional methods and processes used for extracting plant protein isolates and concentrates include alkaline extraction and acid precipitation or ultrafiltration (wet process) and air classification (dry process). The quality of the plant protein compositions produced by these methods is directly dependent on the conditions for isolating and formulating the protein.

Application of an acidic, alkaline, or neutral extraction process directly influences functional properties, e.g., the gelling, foaming or emulsifying properties of the protein compositions obtained, which may make the resulting protein compositions unsuitable for certain applications. It may therefore be necessary to modify the protein compositions, and/or methods of obtaining them, to achieve desired properties for various food applications. There is also a need for providing purified solid plant protein compositions having one or more properties suitable for downstream applications.

Therefore, there exists a need for solid plant protein compositions, and methods of making thereof, that exhibit one or more desired/improved functional and/or organoleptic (sensory) properties, and methods of making and using thereof.

There also exists a need for providing solid plant protein compositions that exhibit one or more suitable/improved properties for downstream applications (e.g., formulation into egg-substitutes as either stand-alone products or components in end products, such as yogurt, cheese, ice-cream, etc.).

### SUMMARY

Solid purified plant protein compositions and methods of making and using thereof are described herein. In some embodiments, the plant protein compositions exhibit one or more desired physical and/or functional properties, e.g., viscosity, gelation capability, gel quality, protein stability, and/or one or more desired organoleptic properties (aroma, taste, etc.).

In some embodiments, the protein can be any plant protein that can be used to prepare plant-based/plant-derived food compositions. In some embodiments, the plant is a pulse or legume.

Exemplary pulses include, but are not limited to, beans, lentils, faba beans, dry peas, chickpeas, cowpeas, bambara beans, pigeon peas, lupins, vetches, adzuki, common beans, fenugreck, long beans, lima beans, runner beans, tepary beans, soybeans, or mucuna beans.

In some embodiments, the pulse is selected from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius.

In some cases, the pulse is mung beans (Vigna radiata or V. radiata) and/or any varietal or cultivar of V. radiata.

In other embodiments, the protein source may be nuts such as almonds and other nuts, seeds such as sesame seeds, sunflower seeds, and other commonly consumed nuts, fruits and seeds.

The solid plant protein compositions described herein further contain a combination of salts. In some embodiments, the combination of salts includes, or is, one or more phosphate salts, one or more metal (e.g., Group I or II) halide salts, and optionally one or more citrate salts. In some embodiments, the combination of salts is one or more phosphate salts and one or more Group I metal halide salts. In some embodiments, the combination of salts is one or more phosphate salts and sodium chloride (NaCl). In some embodiments, the composition contains one or more phosphate salts, one or more metal halide salts, and one or citrate salts. In some embodiments, the composition contains one or more phosphate salts, one or more Group I metal halide salts (e.g., NaCl), and tripotassium citrate.

In some embodiments, the one or more phosphate salts and/or Group I/Group II metal halide salts are present in an amount effective to (1) decrease the viscosity of an aqueous solution of the solid plant protein composition compared to an aqueous solution of the protein without the one or more phosphate salts and one or more Group I/Group II metal halide salts as measured using a viscometer; (2) increase the denaturing temperature of the plant protein compared to the plant protein in the absence of the one or more phosphate salts and/or one or more Group I/Group II metal halide salts, e.g., as measured by DSC; (3) increase the aqueous solubility of the plant protein compared to the plant protein in the absence of the one or more phosphate salts and/or one or more Group I/Group II metal halide salts; or (4) combinations thereof.

In some embodiments, the concentration of the one or more phosphate salts is from about 0.01% to about 5% by weight of the solid plant protein compositions, preferably from about 0.1% to about 2.5%, more preferably from about 0.1% to about 1%.

In some embodiments, the concentration of the one or more Group I/Group II metal halide salts is from about 0.01% to about 5% by weight of the solid plant protein compositions, preferably from about 0.1% to about 2.5%, more preferably from about 0.1% to about 1%.

In some embodiments, the solid plant protein composition further contains one or more citrate salts. In some embodiments, the concentration of the one or more citrate salts is from about 0.01% to about 5% by weight of the solid plant protein compositions, preferably from about 0.1% to about 2.5%, more preferably from about 0.1% to about 1%.

### BRIEF DESCRIPTION OF THE DRAWINGS

FIG. 1 is a bar graph showing ammonia concentration as a function of protein composition (+reaction conditions).

FIG. 2 are differential scanning calorimetry (DSC) profiles for various protein-salt combinations. An aqueous solution of protein alone is used as a control.

FIG. 3 are differential scanning calorimetry (DSC) profiles for various protein-salt combinations. An aqueous solution of protein alone is used as a control.

FIG. 4 is a bar graph showing acceptability means cores for functionality and organoleptic (sensory) properties.

FIG. 5 is a bar graph showing the viscosities for various compositions at days 1, 7, 14, 21, 28, 42, and 56.

### DETAILED DESCRIPTION

It is understood that this disclosure is not limited to the particular methods and experimental conditions described herein, as such methods and conditions may vary. It is also understood that the terminology used herein is for the purpose of describing the particular embodiments only, and is not limiting, since the scope of the present disclosure will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 10%, 9%, 8%, 7%, 6%, 5%, 4%, 3%, 2%, or 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### I. Definitions

As used herein, the singular forms “a,” “an,” and “the” include the plural referents unless the context clearly indicates otherwise.

As used herein, “solid plant protein composition” means a composition containing at least one or more purified plant proteins, one or more phosphate salts, one or more Group I and/or Group II metal halide salts, optionally one or more citrate salts, and optionally transglutaminase (TG), in solid form, e.g., particles, spheres, etc.

As used herein, the term “egg(s)” includes but is not limited to chicken eggs, other bird eggs (such as quail eggs, duck eggs, ostrich eggs, turkey eggs, bantam eggs, goose eggs), and fish eggs such as fish roe. Typical food application comparison is made with respect to chicken eggs.

The term “transglutaminase” refers to an enzyme (R-glutamyl-peptide: amine glutamyl transferase) that catalyzes the acyl-transfer between γ-carboxyamide groups and various primary amines, classified as EC 2.3.2.13. It is used in the food industry to improve texture of some food products such as dairy, meat and cereal products. It can be isolated from a bacterial source, a fungus, a mold, a fish, a mammal and a plant.

The terms “majority” or “predominantly” with respect to a specified component, e.g., small molecule compound, refer to the component having at least 50% by weight of the referenced batch, process stream, food formulation or composition.

Unless indicated otherwise, percentage (%) of ingredients refer to total % by weight typically on a dry weight basis unless otherwise indicated.

The term “aqueous solvent” refers to a water-based fluid. The aqueous solvent can contain salts. The aqueous solvent can contain fluids (e.g., co-solvents) that are miscible in water, such as alcohols, including ethanol.

The terms “volatile small molecule compound” or “small molecule compound” refers to compounds present in the pulse before, during or milling of the pulse.

As used herein “volatile small molecule compound(s)” or “small molecule compound” refers to compound having a molar mass or molecular weight of less than 2,000 Da, less than 1500 Da, less that 1,000 Da, less than 750 Da or less than 500 Da.

As used herein “pulse” refers to legumes that are grown and harvested as food.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Solid Plant Protein Compositions

Solid plant protein compositions are described herein. In some embodiments, the solid plant protein composition contains a purified plant protein, one or more phosphate salts, one or more Group I/Group II metal halide salts, and optionally one or more citrate salts. In some embodiments, the protein is crosslinked by contacting the protein with a cross-linking agent, such as a cross-linking enzyme. In some embodiments, the cross-linking enzyme is introduced simultaneously with the phosphate and metal halide salts or before or after the addition of the salts. In other embodiments, the protein is crosslinked in a downstream process (e.g., during preparation of a food product).

The one or more phosphate salts and/or Group I/Group II metal halide salts are present in an amount effective to reduce the viscosity of a solution (e.g., aqueous) of the purified protein isolate, increase the denaturation temperature of the protein, increase the solubility of the plant protein, or combinations thereof. Moreover, the solid plant protein compositions described herein exhibit improved properties, such as sensory and/or functional (e.g., gelation properties), when formulated into a food product, such as an egg-substitute, compared to the same food product prepared from a protein isolate (i.e., no salts present upstream) in which the salts are added during the formulation of the food product (i.e., downstream).

### A. Plant Protein Source

In some embodiments, suitable sources of plant proteins include plants or plant materials that can be used to prepare food products suitable for human consumption. In some embodiments, the plant or plant material is a pulse. Exemplary pulses include, but are not limited to, beans, lentils, faba beans, dry peas, chickpeas, cowpeas, bambara beans, pigeon peas, lupins, vetches, adzuki, common beans, fenugreek, long beans, lima beans, runner beans, tepary beans, soybeans, or mucuna beans.

In some embodiments, the source of the pulse protein is selected from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius.

In some embodiments, the source of the pulse protein is mung beans (Vigna radiata). In other embodiments, the pulse source may be nuts such as almonds and other nuts, seeds such as sesame seeds, sunflower seeds, and other commonly consumed nuts, fruits and seeds. In some embodiments, the plant protein can be extracted, purified, and isolated as described below.

### B. Phosphate Salts

The solid plant protein composition contains one or more phosphate salts. Suitable phosphate salts include those that, when present in an effective amount: decreases the viscosity of a solution (e.g., aqueous) of the plant protein compared to a solution (e.g., aqueous) of the plant protein without the one or more phosphate salts; increases the denaturing temperature of the plant protein compared to a plant protein composition without the phosphate salts; and/or increases the solubility (e.g., aqueous) of the plant protein compared to a plant protein composition without the phosphate salts, or combinations thereof.

Exemplary phosphate salts include, but are not limited to, sodium, potassium, and/or calcium phosphate salts. Exemplary sodium phosphate salts include, but are not limited to, disodium phosphate (DSP), sodium hexamethaphosphate (SHMP), tetrasodium pyrophosphate (TSPP), and combinations thereof.

In some embodiments, the one or more phosphate salts are present in an amount from about 0.01% to about 5% by weight of the solid plant protein composition, preferably from about 0.1% to about 2.5%, and more preferably from about 0.1% to about 1%. In some embodiments, the one or more phosphate salts are present in an amount from about 0.01% by weight of the solid purified protein isolate to about 1.0% by weight of the solid purified protein isolate. In some embodiments, the concentration of the one or more phosphate salts is about 0.01%, 0.05%, 0.1%, 0.2%, 0.3%, 0.4%, 0.5%, 0.6%, 0.7%, 0.8%, 0.9%, or 1.0%. In some embodiments, the concentration of the one or more phosphate salts is about 0.01%, 0.05%, 0.1%, 0.2%, 0.3%, 0.4%, or 0.5%.

### C. Group I/Group II Metal Halide Salts

The solid compositions described herein further contain one or more Group I and/or Group II metal halide salts. In some embodiments, the one or more additional salts is a Group I metal halide salt. Suitable examples of Group I metal halide salts include, but are not limited, sodium chloride, potassium chloride, and potassium iodide. In some embodiments, the Group I metal halide salt is sodium chloride.

Suitable Group I/Group II salts include those that, when present in an effective amount, do one or more of the following: decreases the viscosity of a solution (e.g., aqueous) of the plant protein composition compared to a solution (e.g., aqueous) of the plant protein composition without the one or more Group I/Group II salts; increases the denaturing temperature of the protein compared to a protein composition without the Group I/Group II salts; and/or increases the solubility (e.g., aqueous) of the protein compared to a protein composition without the Group I/Group II salts; or combinations thereof.

In some embodiments, the one or more Group I/II metal halide salts are present in an amount from about 0.01% to about 5% by weight of the solid plant protein composition, preferably from about 0.1% to about 2.5%, and more preferably from about 0.1% to about 1%. In some embodiments, the one or more phosphate salts are present in an amount from about 0.01% by weight of the solid purified protein isolate to about 1.0% by weight of the solid purified protein isolate. In some embodiments, the concentration of the one or more phosphate salts is about 0.01%, 0.05%, 0.1%, 0.2%, 0.3%, 0.4%, 0.5%, 0.6%, 0.7%, 0.8%, 0.9%, or 1.0%. In some embodiments, the concentration of the one or more phosphate salts is about 0.01%, 0.05%, 0.1%, 0.2%, 0.3%, 0.4%, or 0.5%.

In some embodiments, the one or more additional salts is a Group II metal halide. Suitable examples of Group II metal halide salts include, but are not limited to, calcium chloride and magnesium chloride. Group II metal halide salts can be used at the same concentrations described above with respect to Group I metal halide salts.

In some embodiments, the effect of the combination of the one or more phosphate salts and the one or more Group I/Group II salts on the characteristics described above are additive. In some embodiments, the effect of the combination of the one or more phosphate salts and the one or more Group I/Group II salts on the characteristics described above are more than additive. In some embodiments, the effect of the combination of the one or more phosphate salts and the one or more Group I/Group II salts on the characteristics described above are less than additive.

For example, in some embodiments, the one or more phosphate salts, the Group I/Group II metal halide salts, and combinations thereof are present in amount effective to reduce the viscosity of an aqueous solution of the solid purified protein composition by at least about 1% to about 70% or greater (including all values between 1% and 70%), such as 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater.

In some embodiments, the one or more phosphate salts, the Group I/Group II metal halide salts, and combinations thereof, are present in an amount to reduce the viscosity by about 25 cP, 50 cP, 75 cP, 100 cP, 125 cP, 150 cP, 175 cP, 200 cP, 225 cP, 250 cP, 275 cP, 300 cP, 325 cP, 350 cP, 375 cP, 400 cP, 425 cP, 450 cP, 475 cP, 500 cP, 525 cP, 550 cP, 575 cP, 600 cP, 625 cP, 650 cP, 675 cP, 700 cP, 725 cP, 750 cP, or more.

In some embodiments, the one or more phosphate salts, the Group I/Group II metal halide salts, and combinations thereof, are present in an amount effective to increase the denaturing temperature of the plant protein compared to the absence of the salts. In some embodiments, the one or more phosphate salts, the Group I/Group II metal halide salts, and combinations thereof, are present in an amount effective to increase the denaturing temperature of the protein at least about 0.5° C., 1.0° C., 1.5° C., 2.0° C., 2.5° C., 3.0° C., 3.5° C., 4.0° C., 4.5° C., 5.0° C., 5.5° C., 6.0° C., 6.5° C., 7.0° C., 7.5° C., 8.0° C., or greater. In some embodiments, the increase in the denature temperature is at least about 2.0° C., 2.5° C., 3.0° C., 3.5° C., 4.0° C., 4.5° C., 5.0° C., 5.5° C., 6.0° C., 6.5° C., 7.0° C., 7.5° C., 8.0° C., or greater. For example, the incorporation of a phosphate salt increased the denature temperature from 69.8° C. (water alone) to 77.4° C. (TSPP).

### D. Citrate Salts

In some embodiments, the solid plant protein composition optionally contains one or more citrate salts. Suitable citrate salts include those that, when present in an effective amount: decreases the viscosity of a solution (e.g., aqueous) of the plant protein compared to a solution (e.g., aqueous) of the plant protein without the one or more citrate salts; increases the denaturing temperature of the plant protein compared to a composition of the plant protein without the one or more citrate salts; and/or increases the solubility (e.g., aqueous) of the plant protein compared to a solution (e.g., aqueous) of the plant protein without the one or more citrate salts, or combinations thereof. In some embodiments, suitable citrate salts include, but are not limited to, sodium, potassium, and calcium citrates. Exemplary salts include, but are not limited to, tripotassium citrate (TPC) and trisodium citrate.

In some embodiments, the concentration of the one or more citrate salts is from about 0.01% to about 1.0% (including all values between 0.01% and 1.0%), such as 0.01%, 0.05%, 0.1%, 0.15%, 0.20%, 0.25% 0.30%, 0.35%, 0.40%, 0.45%, 0.5% 0.55%, 0.60%, 0.65%, 0.70%, 0.75%, 0.80%, 0.85%, 0.90%, 0.95%, or 1.0%.

The effect of the presence of the one or more citrate salts in combination with the one or more phosphate salts and the one or more Group I/Group II metal halide salts may be additive, more than additive, and less than additive. In some embodiments, the combination of the one or more citrate salts, one or more phosphate salts, and the one or more Group I/Group II metal halide salts has the effect on the viscosity, solubility, and/or denaturing temperature described above.

With respect to the salt combinations described above, in some embodiments, such salts are taken through downstream process for making food products as described in more detail below and therefore no additional salts are required during downstream processing. In other embodiments, salts, e.g., the same salts described above and/or different salt can be added during downstream processing to prepare the food products described below.

### E. Crosslinking Enzyme

In some embodiments, the plant protein is crosslinked with a crosslinking enzyme. In various embodiments, the cross-linking enzyme is selected from transglutaminase, sortase, subtilisin, tyrosinase, laccase, peroxidase, or lysyl oxidase. In some embodiments, the cross-linking enzyme is transglutaminase.

In some embodiments, the cross-linking enzyme is combined with a purified plant protein and the salts described above and subjected to suitable conditions to activate and deactivate enzyme. Such conditions are known in the art and are described in more detail below. In some embodiments, the amount of cross-linking enzyme is from about 0.0001% to about 0.1%, from about 0.001% to about 0.05%, or from about 0.001% to about 0.0125% by the weight of the composition.

In some embodiments, the plant protein may be contacted with a protein modifying enzyme such as papain, pepsin, peptidase, rennet, coagulating enzymes or sulfhydryl oxidase to modify the structure of the plant protein(s). Conditions for reacting the protein modifying enzyme with the protein are known in the art.

### III. Methods for Extracting, Purifying, and Isolating Plant Proteins

### A. Plant Preparation

Methods for extracting, purifying, and isolating plant proteins are known in the art. In some embodiments, the method for extracting the plant protein from the source involves preparing the plant source, for example, dehulling the plant source in embodiments where the source is a pulse. Alternatively, the pulse source can remain hulled. In other embodiments, the source is a plant material that does not contain a hull. In some embodiments, the pulse is dehulled by contacting the pulse with a solvent for a desired amount of time. In one embodiment, the solvent used for dehulling the pulse is water, ethanol, oil, and/or other solvents. In some embodiments, salts such as sodium salts, potassium salts, ammonium salts or other salts can be added to the solvent.

In some embodiments, the method further includes heating (roasting) the plant source. Such heating (roasting) methods, for example for pulses, are described in WO2022/087130 to Eat Just, Inc. In some embodiments, the heat treatment involves or includes exposing the plant material, in the absence of solvent, to one or more heating zones for a desired amount of time. The temperature of one heating zone may be different than the temperature of another heating zone. In some embodiments, the plant material is exposed to steam in the one or more heating zones. In some embodiments, after treatment in the one or more heating zones, the plant material is exposed to a cooling zone to cool the heat-treated pulse to a desired temperature.

In some embodiments, after the plant material is heated (roasted), the heat-treated plant material is milled. In some embodiments, the heat-treated plant material is dry milled. In other embodiments, the heat-treated plant material is wet-milled. In some embodiments, wet milling includes: (1) incubating a plant material in an aqueous solvent to prepare a hydrated plant material and (2) milling the hydrated plant material to prepare the wet-milled plant material; also referred to as a wet-milled plant material flour.

In some embodiments, the aqueous solvent can be neat water or include or contain salts. Exemplary salts include, but are not limited to, NaCl, NaHCO₃, Na₂CO₃, Na₂SO₄, NaH₂PO₄, Na₂HPO₄, Na₃PO₄. Na₂SO₄, KCl, KHCO₃, K₂CO₃, Na₂SO₄, KH₂PO₄, K₂HPO₄, K₃PO₄. K₂SO₄ sodium citrate, sodium acetate, potassium citrate, and potassium acetate. In various embodiments, the salt is selected from sodium chloride, sodium sulfate, sodium phosphate, ammonium sulfate, ammonium phosphate, ammonium chloride, potassium chloride, potassium sulfate, or potassium phosphate. In some embodiments, the salt is NaCl. In some embodiments, the aqueous solution does not contain a salt.

### B. Protein Extraction and Separation

Methods for extracting plant protein from a plant material (e.g., milled) containing plant proteins are known in the art. In some embodiments, the plant proteins are extracted from the plant material in an aqueous solution at a pH of from about 1 to about 10 to produce a protein rich fraction containing extracted plant proteins. In some embodiments, the aqueous solution has a pH of from about 4 to about 9. In some embodiments, the aqueous solution has a pH of from about 6 to about 10. In some embodiments, the aqueous solution has a pH of about 7 to about 9. In some embodiments, the aqueous solution has a pH of about 8. The pH of the slurry may be adjusted with, e.g., a food-grade 50% sodium hydroxide solution to reach the desired extraction pH.

Following extraction, the protein rich fraction may be separated from the slurry, for example, in a solid/liquid separation unit, consisting of a decanter and a disc-stack centrifuge. The protein rich fraction may be centrifuged at a low temperature, e.g., between 3-10° C. In some cases, the protein rich fraction is precipitated, collected and the pellet is resuspended. The process may be repeated, and the combined protein rich fractions filtered through a Nylon mesh.

In some embodiments, the methods may optionally include reducing or removing a fraction containing carbohydrates (e.g., starches) or a carbohydrate-rich protein isolate, post extraction.

In other embodiments, the protein rich fraction may be separated by ultrafiltration. Methods for ultrafiltration are described in WO2021/174017 by Eat Just, Inc.

In some embodiments, the protein rich fraction, retentate fraction, or plant protein isolate may be subjected to a carbon adsorption step to remove non-protein, off-flavor components, and additional fibrous solids from the protein extraction. This carbon adsorption step leads to a clarified protein extract.

### IV. Method for Preparing Solid Plant Protein Compositions

In some embodiments, the plant protein is extracted, purified, and isolated as described above. In some embodiments, the plant protein is a pulse protein, which can be extracted, purified, and isolated as described in Example 1.

In some embodiments, a (solid, e.g., pelletized) purified plant protein is resuspended in water to form a suspension or slurry. In some embodiments, the total solids content of the protein in the suspension/slurry is from about 10% to about 20% by weight. However, the solids content can be varied higher or lower as needed.

A mixture of one or more phosphate salts, one or more Group I/Group II metal halide salts, and optionally one or more citrate salts, are added to the suspension/slurry. In some embodiments, a crosslinking enzyme, such as transglutaminase (TG), is added, either alone or as part of the mixture of salts. If TG is added, the suspension/slurry may be incubated, for example, at a temperature from about 45° C. to about 60° C., preferably from about 50° C. to about 55° C. for a period of time from about 5 minutes to about 60 minutes, preferably from about 10 minutes to about 50 minutes, preferably from about 10 to about 45 minutes, more preferably from about 10 minutes to about 20 minutes to activate TG. Following incubation, the suspension/slurry is pasteurized by increasing the temperature, for example, to about 65° C. to about 80° C., preferably from about 65° C. to about 75° C., more preferably from about 70° C. to about 75° C. from a period of time from about 0.5 minutes to about 15 minutes, preferably from about 1 minutes to about 10 minutes, or preferably from about 2 to about 8 minutes to deactivate or “kill” the crosslinking enzyme, e.g., TG.

Following pasteurization, the suspension/slurry is dried to form the solid plant protein composition. The suspension/slurry can be dried using a variety of techniques known in the art including, but not limited to, freeze drying, spray drying, spray freeze drying, and supercritical fluid drying. In some embodiments, the suspension/slurry is spray dried to form a solid purified plant protein composition. In some embodiments, the residual water is less than about 10%, 9%, 8%, 7%, 6%, 5%, 4%, 3%, 2%, or 1% by weight of the composition.

The solid purified pulse protein compositions described herein can be packaged using techniques known in the art and sold as a raw material or proprietary ingredient for various downstream applications/products.

### V. Characteristics of Plant Protein Compositions

### A. Compositional Characteristics

The solid plant protein compositions described herein are edible and exhibit one or more desirable food/food product qualities including, but not limited to, high protein content, reduced retention of small molecular weight non-protein species (including mono and disaccharides), reduced retention of oils and lipids, improved structural/structure building properties such as high gel strength and gel elasticity, improved sensory properties, and selective enrichment of certain proteins, e.g., highly functional 8s globulin/beta conglycinin proteins.

In some embodiments, improved properties are observed in a downstream or final product prepared from the solid purified plant protein compositions, such as a liquid egg-substitute, compared to a liquid egg substitute wherein the mixture of salts are added during formulation of the downstream product.

In various embodiments, the solid plant protein composition contains plant protein at a concentration from about 50% to about 90% by weight of the solid plant protein composition, for example from about 50% to about 60%, from about 60% to about 70%, from about 70% to about 80%, or from about 80% to about 90%, or more. In some embodiments, the plant protein composition contains about 80%, 81%, 82%, 83%, 84%, 85%, 86%, 87%, 88%, 89%, 90%, 91%, 92%, 93%, 94%, 95%, 96%, 97%, 98%, or 99% or more plant proteins. In some embodiments, at least about 60% by weight of the plant protein composition is plant protein(s). In some embodiments, at least about 65%, 70%, 75%, 80%, 85%, 90%, or 95% or more by weight of the plant protein composition is plant protein(s).

In some embodiments in which the plant protein in the solid plant protein composition is mung bean protein, at least about 10%, 15%, 20%, 25%, 30%, 35%, 40%, 45%, 50%, 55%, 60%, 65%, 70%, 75%, 80%, 85%, or greater than 85% by weight of the composition is 8s globulin/beta-conglycinin. In other embodiments, about 60% to about 80%, about 65% to about 85%, about 70% to about 90%, or about 75% to about 95% by weight of the mung bean protein composition contains or includes mung bean 8s globulin/beta-conglycinin. In some embodiments, the mung bean protein composition is reduced in the amount of 11s globulin relative to whole mung bean or mung bean flour. In some embodiments, the amount of 11s globulin is less than 10%, 8%, 8%, 7%, 6%, 5%, 4%, 3%, 2%, or 1% of the protein in the mung bean protein composition.

In some embodiments, the solid plant protein composition includes or contains about 0.05 to 0.1%, 1% to 10%, 2% to 9%, 3% to 8%, or 4% to 6% of carbohydrates (e.g., starch, polysaccharides, fiber) derived from the plant source of the plant protein. In some embodiments, the solid plant protein composition contains less than about 10%, 9%, 8%, 7%, 6%, or 5% of carbohydrates derived from the plant source of the protein. In some embodiments, the solid plant protein composition contains about 9%, 8%, 7%, 6%, 5%, 4%, 3%, 2%, or about 1% of carbohydrates derived from the plant source of the plant protein.

In some embodiments, the solid plant protein composition contains about 1% to 10%, 2% to 9%, 3% to 8%, or 4% to 6% of ash derived from the plant source of the protein. In some embodiments, the solid plant protein composition contains less than about 10%, 9%, 8%, 7%, 6%, or 5% of ash derived from the plant source of the protein. In some embodiments, the solid plant protein composition contains about 9%, 8%, 7%, 6%, 5%, 4%, 3%, 2%, or about 1% of ash derived from the plant source of the protein.

In some embodiments, the solid plant protein composition contains about 1% to 10%, 2% to 9%, 3% to 8%, or 4% to 6% of fats derived from the plant source of the protein. In some embodiments, the solid plant protein composition contains less than about 10%, 9%, 8%, 7%, 6%, or 5% of fats derived from the plant source of the protein. In some embodiments, the solid plant protein composition contains about 9%, 8%, 7%, 6%, 5%, 4%, 3%, 2%, or about 1% of fats derived from the plant source of the protein.

### B. Mechanical Characteristics

In various embodiments, the methods discussed above or herein produce a solid plant protein composition have various desirable mechanical properties. In some embodiments, the solid plant protein compositions, or solutions (e.g. aqueous) thereof, have a storage modulus of from 25 Pa to 500 Pa at a temperature between 90° C. and 95° C., as measured by dynamic oscillatory rheology using a rheometer equipped with a flat parallel plate geometry of 40 mm in which the measured solid pulse protein isolate composition contains 12% w/w protein and the storage modulus is recorded under 0.1% strain conditions at a constant angular frequency of 10 rad/s. In various embodiments, the methods discussed above or herein produce a solid plant protein composition having a storage modulus of less than 50 Pa at a temperature between 90° C. and 95° C., as measured by dynamic oscillatory rheology using a rheometer equipped with a flat parallel plate geometry of 40 mm in which the measured solid pulse protein isolate composition contains 12% w/w protein and the storage modulus is recorded under 0.1% strain conditions at a constant angular frequency of 10 rad/s. In some embodiments, the plant protein composition exhibiting the above characteristics is a mung bean protein composition containing one or more phosphate salts, one or more citrate salts, and one or more additional salts (NaCl).

In various embodiments, the methods discussed above or herein produce a solid plant protein composition having a linear viscoelastic region of from 25 Pa to 1500 Pa at up to 10% strain, as measured by dynamic oscillatory rheology using a rheometer equipped with a flat parallel plate geometry of 40 mm in which the measured solid plant protein composition contains 12% w/w protein and the strain is carried out at a constant frequency of 10 rad/s at 50° C. In various embodiments, the methods discussed above or herein produce a solid plant protein composition having a linear viscoelastic region of less than 1000 Pa at up to 10% strain, as measured by dynamic oscillatory rheology using a rheometer equipped with a flat parallel plate geometry of 40 mm in which the measured solid plant protein composition contains 12% w/w protein and the strain is carried out at a constant frequency of 10 rad/s at 50° C. In some embodiments, the methods produce a s solid plant protein composition having a linear viscoelastic region of less than 500 Pa at up to 10% strain, or a linear viscoelastic region of less than 200 Pa at up to 10% strain. In some embodiments, the plant protein composition exhibiting the above characteristics is a mung bean protein composition containing one or more phosphate salts, one or more citrate salts, and one or more additional salts (NaCl).

### C. Allergen Characteristics

In some embodiments, the solid plant protein composition provided herein have a reduced allergen content. In some embodiments, the reduced allergen content is relative to the allergen content of the plant source of the isolate. The solid plant protein composition or a composition containing the solid plant protein composition may be animal-free, dairy-free, soy-free and gluten-free. Adverse immune responses such as hives or rash, swelling, wheezing, stomach pain, cramps, diarrhea, vomiting, dizziness and even anaphylaxis presented in subjects who are typically allergic to eggs may be averted. Further, the solid plant protein composition or a composition containing the solid plant protein composition may not trigger allergic reactions in subjects based on milk, eggs, soy and wheat allergens. Accordingly, in some embodiments, the solid plant protein composition or a composition containing the solid plant protein composition is substantially free of allergens.

### D. Dietary Characteristics

Dietary anti-nutritional factors are chemical substances that can adversely impact the digestibility of protein, bioavailability of amino acids and protein quality of foods (Gilani et al., 2012). In some embodiments, the solid plant protein composition provided herein have reduced amounts of anti-nutritional factors. In some embodiments, the reduced amount of anti-nutritional factors is relative to the content of the plant source of the plant proteins. In some embodiments, the reduced anti-nutritional factor is selected from tannins, phytic acid, hemagglutinins (lectins), polyphenols, trypsin inhibitors, α-amylase inhibitors, lectins, protease inhibitors, and combinations thereof.

In various embodiments, the solid plant protein composition is either free from environmental contaminants, below the level of detection of 0.1 ppm, or present at levels that pose no toxicological significance.

### VI. Uses of Solid Plant Protein Compositions

### A. Functional and Sensory (Organoleptic) Characteristics

In various embodiments, the solid plant protein compositions exhibit desirable functional characteristics such as emulsification, water binding, foaming and gelation properties comparable to natural food or food product, such as an egg. In various embodiments, the solid plant protein composition exhibits one or more functional or sensory properties advantageous for use in food compositions. The functional properties may include, but are not limited to, crumb density, structure/texture, elasticity/springiness, coagulation, binding, moisturizing, mouthfeel, leavening, aeration/foaming, creaminess, and emulsification of the food composition.

Mouthfeel is a concept used in the testing and description of food products. Products made using solid plant protein composition discussed herein can be assessed for mouthfeel. Products, e.g., baked goods, made using the solid plant protein composition have mouthfeel that is similar to products made with natural eggs. In some embodiments, the mouthfeel of the products made using the solid plant protein composition is superior to the mouthfeel of previously known or attempted egg substitutes, e.g., bananas, modified whey proteins, or Egg Beaters™ as well as egg-substitutes prepared from a pulse protein isolate wherein the mixture of salts (and optionally TG) is incorporated downstream during formulation of the food or food product.

Examples of properties which may be included in a measure of mouthfeel include:
(1) cohesiveness-degree to which the sample deforms before rupturing when biting with molars;
(2) density-compactness of cross section of the sample after biting completely through with the molars;
(3) dryness-degree to which the sample feels dry in the mouth; fracturability-force with which the sample crumbles, cracks or shatters (fracturability encompasses crumbliness, crispiness, crunchiness and brittleness);
(4) graininess-degree to which a sample contains small grainy particles, may be seen as the opposite of smoothness;
(5) gumminess-energy required to disintegrate a semi-solid food to a state ready for swallowing;
(6) hardness-force required to deform the product to given distance, i.e., force to compress between molars, bite through with incisors, compress between tongue and palate; heaviness-weight of product perceived when first placed on tongue;
(7) moisture absorption-amount of saliva absorbed by product;
(8) moisture release-amount of wetness/juiciness released from sample;
(9) mouthcoating-type and degree of coating in the mouth after mastication (for example, fat/oil);
(10) roughness-degree of abrasiveness of product's surface perceived by the tongue;
Slipperiness: degree to which the product slides over the tongue;
(11) smoothness-absence of any particles, lumps, bumps, etc., in the product;
(12) uniformity-degree to which the sample is even throughout (homogeneity);
(13) uniformity of bite: evenness of force through bite;
(14) uniformity of chew-degree to which the chewing characteristics of the product are even throughout mastication;
(15) viscosity-force required to draw a liquid from a spoon over the tongue; and
(16) wetness-amount of moisture perceived on product's surface.

The solid plant protein composition discussed herein may also have one or more functional properties alone or when incorporated into a food composition. Such functional properties may include, but are not limited to, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color. In some embodiments, at least one functional property of the solid plant protein composition is similar or equivalent to the corresponding functional property of the source of the plant protein.

In some embodiments, at least one functional property of the solid plant protein composition (alone or when incorporated into a food composition) is similar or equivalent to the corresponding functional property of a reference food product, such as, for example, an egg (liquid, scrambled, or in patty or folded form), a cake (e.g., pound cake, yellow cake, or angel food cake), a cream cheese, a pasta, an emulsion, a confection, an ice cream, a custard, milk, a deli meat, chicken (e.g., chicken nuggets), a yogurt, or a coating. In some embodiments, the solid plant protein composition, either alone or when incorporated into a composition, is capable of forming a gel under heat or at room temperature.

In some embodiments, the solid plant protein composition described herein exhibit improved properties, such as sensory and/or gelation properties, when formulated into a food product, such as an egg-substitute, compared to the same food product prepared from a protein composition in which the salts are added during the formulation of the food product. For example, as shown in FIG. 2, a liquid egg substitute prepared from the solid plant protein composition described herein exhibit improved functionality, texture, flavor, and viscosity compared to the control (salts added downstream during liquid egg substitute formulation).

In some embodiments, the solid plant protein composition discussed herein may have modulated organoleptic properties of one or more of the following characteristics: astringent, beany, bitter, burnt, buttery, nutty, sweet, sour, fruity, floral, woody, earthy, beany, spicy, metallic, sweet, musty, grassy, green, oily, vinegary, neutral and bland flavor or aromas. In some embodiments, the solid plant protein composition exhibit modulated organoleptic properties such as a reduction or absence in one or more of the following: astringent, beany, bitter, burnt, buttery, nutty, sweet, sour, fruity, floral, woody, earthy, beany, spicy, metallic, sweet, musty, grassy, green, oily, vinegary neutral and bland flavor or aromas.

### VII. Food Products Containing Solid Plant Protein Compositions

The solid plant protein composition described herein may be incorporated into a food product along with one or more other edible ingredients. In some cases, the solid plant protein composition may be used as a direct protein replacement of animal—or vegetable-based protein in a variety of conventional food and beverage products across multiple categories. In some embodiments, the use levels range from about 3% to about 90% w/w of the final product.

In some embodiments, the solid plant protein composition as described above is used as a supplement to existing protein in food products. In some embodiments, the protein may be crosslinked downstream rather than upstream. In those embodiments, the protein can be crosslinked using a crosslinking agent, such as a crosslinking enzyme. In various embodiments, the cross-linking enzyme is selected from transglutaminase, sortase, subtilisin, tyrosinase, laccase, peroxidase, or lysyl oxidase. In some embodiments, the cross-linking enzyme is transglutaminase. In any of the various embodiments of the food compositions, the plant protein may be contacted with a protein modifying enzyme such as papain, pepsin, peptidase, rennet, coagulating enzymes or sulfhydryl oxidase to modify the structure of the pulse proteins.

Exemplary food products in which the solid plant protein composition can be used are discussed below.

The solid plant protein composition provided herein are suitable for various food applications and can be incorporated into, e.g., edible egg-free emulsion, egg analog, egg-free scrambled eggs, egg-free patty, egg-free pound cake, egg-free angel food cake, egg-free yellow cake, egg-free cream cheese, egg-free pasta dough, egg-free custard, egg-free ice cream, dairy-free yogurt, and dairy-free milk. The solid plant protein compositions can also be used as replacement ingredients in various food applications including but not limited to meat substitutes, egg substitutes, baked goods and fortified drinks.

In various embodiments, one or more solid plant protein composition can be incorporated into multiple food products, including liquid and patty scrambled egg substitutes to a desired level of emulsification, water binding and gelation. In an embodiment, a functional egg replacement product contains solid plant protein composition (8-15%), and one or more of: oil (10%), hydrocolloid, preservative, and optionally flavors, water, lecithin, xanthan, sodium carbonate, and black salt. In some embodiments, the solid plant protein composition is incorporated in an egg substitute composition. In some such embodiments, the organoleptic property of the solid plant protein composition (e.g., a flavor or an aroma) is similar or equivalent to a corresponding organoleptic property of an egg. The egg substitute product may exhibit at least one functional property (e.g., emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, or color) that is similar or equivalent to a corresponding functional property of an egg. In addition to the solid plant protein composition, the egg substitute product may include one or more of iota-carrageenan, gum arabic, konjac, xanthan gum, or gellan.

In some embodiments, the solid plant protein composition is incorporated in an egg-free cake, such as a pound cake, a yellow cake, or an angel food cake. In some such embodiments, at least one organoleptic property (e.g., a flavor or an aroma) of the egg-free cake is similar or equivalent to a corresponding organoleptic property of a cake containing eggs. The egg-free cake may exhibit at least one functional property similar or equivalent to a corresponding functional property of a cake containing eggs. The at least one function property may be, for example, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, color, or a combination thereof. In some embodiments in which the solid plant protein composition is included in an egg-free pound cake, a peak height of the egg-free pound cake is at least 90% of the peak height of a pound cake containing eggs.

In some embodiments, the solid plant protein composition is incorporated into an egg-free cake mix or an egg-free cake batter. In some such embodiments, the egg-free cake mix or batter has at least one organoleptic property (e.g., a flavor or aroma) that is similar or equivalent to a corresponding organoleptic property of a cake mix or batter containing eggs. The egg-free cake mix or batter may exhibit at least one functional property similar or equivalent to a corresponding functional property of a cake batter containing eggs. The at least one functional property may be, for example, one or more of emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, color, or a combination thereof. In some embodiments in which the solid pulse protein isolate composition is included in an egg-free pound cake batter, a specific gravity of the egg-free pound cake batter is 0.95-0.99.

In some cases, increased functionality is associated with the solid plant protein composition in a food product. For instance, food products produced with the solid plant protein composition discussed herein may exhibit increased functionality in dome or crack, cake resilience, cake cohesiveness, cake springiness, cake peak height, specific gravity of batter, center doming, center crack, browning, mouthfeel, spring-back, off flavors or flavor.

In some embodiments, the solid plant protein composition is included in a cream cheese, a pasta dough, a pasta, a milk, a custard, a frozen dessert (e.g., a frozen dessert comprising ice cream), a deli meat, or chicken (e.g., chicken nuggets).

In some embodiments, the solid plant protein composition is incorporated into a food or beverage composition, such as, for example, an egg substitute, a cake (e.g., a pound cake, a yellow cake, or an angel food cake), a cake batter, a cake mix, a cream cheese, a pasta dough, a pasta, a custard, an ice cream, a milk, a deli meat, or a confection. The food or beverage product may provide sensory impressions similar or equivalent to the texture and mouthfeel that replicates a reference food or beverage composition. In some embodiments, before being included in a food or beverage composition, the solid plant protein composition is further processed in a manner that depends on a target application for the solid plant protein composition. For example, the solid plant protein composition may be diluted in a buffer to adjust the pH to a pH appropriate for the target application. As another example, the solid plant protein composition may be concentrated for use in the target application. As yet another example, the solid plant protein composition may be dried for use in the target application. Various examples of food products containing the solid plant protein composition discussed herein are provided below.

### A. Scrambled Egg Analog

In some embodiments, the solid plant protein composition is incorporated into a scrambled egg analog in which the plant protein (e.g., mung bean protein) has been contacted with transglutaminase (or other cross-linking enzyme) to provide advantageous textural, functional and organoleptic properties. Food processing methods employing transglutaminases are known in the art. The plant protein can be contacted with cross-linking enzyme during preparation of the solid plant protein composition (simultaneous with the addition of phosphate, citrate, and additional salts; prior to addition of the salt, or after the addition of the salts). Alternatively, the plant protein can be contacted with the cross-linking enzyme during downstream use of the solid plant protein composition (e.g., after formation of the solid plant protein composition and during formulation of a food product).

In some embodiments, the transglutaminase is microencapsulated when utilized in the egg analogs provided herein. Microencapsulation of transglutaminase enzyme in such egg mimetic emulsions maintains a stable emulsion by preventing contact of the protein substrate with the transglutaminase enzyme. A cross-linking reaction is initiated upon heating to melt the microencapsulating composition. In some embodiments, the transglutaminase is immobilized on inert porous beads or polymer sheets and contacted with the egg mimetic emulsions.

In various embodiments, the scrambled egg analog contains a solid plant protein composition described herein, along with one or more of the following components: water, disodium phosphate and oil. In some embodiments, the scrambled egg analog further contains NaCl. In a particular embodiment, the scrambled egg analog contains: Protein Solids: 11.3 g, Water: 81.79 g, Disodium phosphate: 0.4 g, Oil: 6.2 g, NaCl: 0.31 g (based on total weight of 100 g) wherein the protein solids are or were contacted with between 0.001% and 0.0125% of transglutaminase.

### B. Vegan Patty

Solid plant protein composition (e.g., mung bean protein) can be used as the sole gelling agent in a formulated vegan patty. In some embodiments, a hydrocolloid system containing iota-carrageenan and gum arabic enhances native gelling properties of the solid plant protein composition in a formulated patty. In other embodiments, a hydrocolloid system containing high-acyl and low-acyl gellan in a 1.5:1 ratio enhances native gelling properties of the solid plant protein composition in a formulated patty. In further embodiments, a hydrocolloid system containing konjac and xanthan gum enhances native gelling properties of the solid plant protein composition in a formulated patty.

### C. Egg-Free Emulsion

In another embodiment, solid plant protein compositions (e.g., mung bean protein) are included in an edible egg-free emulsion. In some embodiments, the emulsion contains one or more additional components selected from water, oil, fat, hydrocolloid, and starch. In some embodiments, at least or about 60-85% of the edible egg-free emulsion is water. In some embodiments, at least or about 10-20% of the edible egg-free emulsion is the solid plant protein composition. In some embodiments, at least or about 5-15% of the edible egg-free emulsion is oil or fat. In some embodiments, at least or about 0.01-6% of the edible egg-free emulsion is the hydrocolloid fraction or starch. In some embodiments, the hydrocolloid fraction contains high-acyl gellan gum, low-acyl gellan gum, iota-carrageenan, gum arabic, konjac, locust bean gum, guar gum, xanthan gum, or a combination of one or more gums thereof. In some embodiments, the emulsion further contains one or more of: a flavoring, a coloring agent, an antimicrobial, a leavening agent, and salt. In some embodiments, the emulsion further contains one or more salts such as those described above or different salts.

In one embodiment, the edible egg-free emulsion has a pH of about 5.6 to 6.8. In some embodiments, the edible egg-free emulsion contains water, a solid plant protein composition as described herein, optionally an enzyme that modifies a structure of the solid plant protein composition, and oil or fat. In some embodiments, the enzyme contains a transglutaminase or proteolytic enzyme. In some embodiments, at least or about 70-85% of the edible egg-free emulsion is water. In some embodiments, at least or about 7-15% of the edible egg-free emulsion is the solid plant protein composition. In some embodiments, at least or about 0.0005-0.0025% (5-25 parts per million) of the edible egg-free emulsion is the enzyme that modifies the structure of the solid plant protein composition. In some embodiments, at least or about 5-15% of the edible egg-free emulsion is oil or fat.

### D. Baked Cake Mixes and Batters

In another embodiment, solid plant protein compositions (e.g., mung bean protein) are included in one or more egg-free cake mixes, suitable for preparing one or more egg-free cake batters, from which one or more egg-free cakes can be made. In some embodiments, the egg-free cake mix contains grain flour, for example, wheat flour or other grain flour, sugar, and a solid plant protein composition. In some embodiments, the egg-free cake mix further contains one or more additional components selected from: cream of tartar, disodium phosphate, baking soda, and a pH stabilizing agent. In some embodiments, the flour contains cake flour.

In another embodiment, solid plant protein compositions (e.g., mung bean protein) are included in an egg-free cake batter containing an egg-free cake mix described above, and water. In some embodiments, the egg-free cake batter is an egg-free pound cake batter, an egg-free angel food cake batter, or an egg-free yellow cake batter. In some embodiments, the egg-free cake batter has a specific gravity of 0.95-0.99.

In one embodiment, an egg-free pound cake mix contains wheat flour, sugar, and a pulse protein isolate. In some embodiments, the flour contains cake flour. In some embodiments, the egg-free pound cake mix further contains oil or fat. In some embodiments, the oil or fat contains butter or shortening. In some embodiments, at least or about 25-31% of the egg-free pound cake batter is flour. In some embodiments, at least or about 25-31% of the egg-free pound cake batter is oil or fat. In some embodiments, at least or about 25-31% of the egg-free pound cake batter is sugar. In some embodiments, at least or about 6-12% of the egg-free pound cake batter is the pulse protein isolate. In some embodiments, the batter further contains disodium phosphate or baking soda.

In an embodiment, an egg-free pound cake batter contains an egg-free pound cake mix described above, and further contains water. In some embodiments, the egg-free pound cake batter contains about four parts of the egg-free pound cake mix; and about one part water. In some embodiments, at least or about 20-25% of the egg-free pound cake batter is wheat flour. In some embodiments, at least or about 20-25% of the egg-free pound cake batter is oil or fat. In some embodiments, at least or about 20-25% of the egg-free pound cake batter is sugar. In some embodiments, at least or about 5-8% of the egg-free pound cake batter is the solid plant protein composition. In some embodiments, at least or about 18-20% of the egg-free pound cake batter is water.

In an embodiment, an egg-free angel food cake mix contains wheat flour, sugar, and a solid plant protein composition. In some embodiments, at least or about 8-16% of the egg-free angel food cake mix is wheat flour. In some embodiments, at least or about 29-42% of the egg-free angel food cake mix is sugar. In some embodiments, at least or about 7-10% of the egg-free angel food cake mix is the solid plant protein composition. In some embodiments, the egg-free angel food cake mix further contains cream of tartar, disodium phosphate, baking soda, or a pH stabilizing agent. In some embodiments, the wheat flour contains cake flour. Also provided herein is an egg-free angel food cake batter containing an egg-free angel food cake mix described above, and water.

In an embodiment, an egg-free yellow cake mix contains wheat flour, sugar, and a solid plant protein composition. In some embodiments, at least or about 20-33% of the egg-free yellow cake mix is wheat flour. In some embodiments, at least or about 19-39% of the egg-free yellow cake mix is sugar. In some embodiments, at least or about 4-7% of the egg-free yellow cake mix is the solid plant protein composition. In some embodiments, the egg-free yellow cake mix further contains one or more of baking powder, salt, dry milk, and shortening. Also provided herein is an egg-free yellow cake batter containing an egg-free yellow cake mix described above, and water.

Sensory quality parameters of cakes made with the solid plant protein composition are characterized as fluffy, soft, airy texture. The peak height is measured to be 90-110% of pound cake containing eggs. The specific gravity of cake batter with the purified pulse protein isolate is 0.95-0.99, similar to that of cake batter with whole eggs of 0.95-0.96.

### E. Cream Cheese Analog

In another embodiment, solid plant protein compositions (e.g., mung bean protein) are included in an egg-free cream cheese. In some embodiments, the egg-free cream cheese contains one or more additional components selected from water, oil or fat, and hydrocolloid. In some embodiments, at least or about 75-85% of the egg-free cream cheese is water. In some embodiments, at least or about 10-15% of the egg-free cream cheese is the solid plant protein composition. In some embodiments, at least or about 5-10% of the egg-free cream cheese is oil or fat. In some embodiments, at least or about 0.1-3% of the egg-free cream cheese is hydrocolloid. In some embodiments, the hydrocolloid comprises xanthan gum or a low-methoxy pectin and calcium chloride system. In some embodiments, the egg-free cream cheese further contains a flavoring or salt. In some embodiments, one or more characteristics of the egg-free cream cheese is similar or equivalent to one or more corresponding characteristics of a cream cheese containing eggs. In some embodiments, the characteristic is a taste, a viscosity, a creaminess, a consistency, a smell, a spreadability, a color, a thermal stability, or a melting property. In some embodiments, the characteristic comprises a functional property or an organoleptic property. In some embodiments, the functional property is selected from emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, and/or color. In some embodiments, the organoleptic property is a flavor or an odor.

F. Egg-free pasta dough

In another embodiment, solid plant protein compositions (e.g., mung bean protein) are included in an egg-free pasta dough. In some embodiments, the egg-free pasta dough contains one or more additional components selected from grain flour, oil or fat, and water. In some embodiments, the flour contains semolina flour. In some embodiments, the oil or fat contains extra virgin oil. In some embodiments, the egg-free pasta dough further contains salt. Also provided herein is an egg-free pasta made from an egg-free pasta dough described above. In some embodiments, the egg-free pasta is fresh. In some embodiments, the egg-free pasta is dried. In some embodiments, one or more characteristics of the egg-free pasta is similar or equivalent to one or more corresponding characteristics of a pasta containing eggs. In some embodiments, the one or more characteristics include chewiness, density, taste, cooking time, shelf life, cohesiveness, or stickiness. In some embodiments, the one or more characteristics include a functional property or an organoleptic property. In some embodiments, the functional property includes emulsification, water binding capacity, foaming, gelation, crumb density, structure forming, texture building, cohesion, adhesion, elasticity, springiness, solubility, viscosity, fat absorption, flavor binding, coagulation, leavening, aeration, creaminess, film forming property, sheen addition, shine addition, freeze stability, thaw stability, and/or color. In some embodiments, the organoleptic property is a flavor or an odor.

### G. Plant-Based Milk

In another embodiment, solid plant protein compositions (e.g., mung bean protein) are included in a plant-based milk. In some embodiments, the plant-based milk contains one or more additional components selected from water, oil or fat, and sugar. In some embodiments, at least or about 5% of the plant-based milk is the solid plant protein composition. In some embodiments, at least or about 70% of the plant-based milk is water. In some embodiments, at least or about 2% of the plant-based milk is oil or fat. In some embodiments, the plant-based milk further contains one or more of: disodium phosphate, soy lecithin, and trace minerals. In particular embodiments, the plant-based milk is lactose-free. In other particular embodiments, the plant-based milk does not contain gums or stabilizers.

### H. Egg-Free Custard

In another embodiment, solid plant protein compositions (e.g., mung bean protein) are included in an egg-free custard. In some embodiments, the egg-free custard contains one or more additional components selected from cream and sugar. In some embodiments, at least or about 5% of the egg-free custard is the solid plant protein composition. In some embodiments, at least or about 81% of the egg-free custard is cream. In some embodiments, at least or about 13% of the egg-free custard is sugar. In some embodiments, the egg-free custard further contains one or more of: iota-carrageenan, kappa-carrageenan, vanilla, and salt. In some embodiments, the cream is heavy cream.

### I. Egg-Free Ice Cream

In another embodiment, solid plant protein compositions (e.g., mung bean protein) are included in an egg-free ice cream. In some embodiments, the egg-free ice cream is a soft-serve ice cream or a regular ice cream. In some embodiments, the egg-free ice cream contains one or more additional components selected from cream, milk, and sugar. In some embodiments, at least or about 5% of the egg-free ice cream is the solid plant protein composition. In some embodiments, at least or about 41% of the egg-free ice cream is cream. In some embodiments, at least or about 40% of the egg-free ice cream is milk. In some embodiments, at least or about 13% of the egg-free ice cream is sugar. In some embodiments, the egg-free ice cream further contains one or more of iota carrageenan, kappa carrageenan, vanilla, and salt. In some embodiments, the cream is heavy cream. In some embodiments, the milk is whole milk. In particular embodiments, the egg-free ice cream is lactose-free. In some embodiments, the egg-free ice cream does not contain gums or stabilizers. In some embodiments, the egg-free ice provides a traditional mouthfeel and texture of an egg-based ice cream but melts at a slower rate relative to an egg-based ice cream.

### J. Fat Reduction Shortening System (FRSS)

In another embodiment, solid plant protein compositions (e.g., mung bean protein) are included in a fat reduction shortening system. In some embodiments, the FRSS contains one or more additional components selected from water, oil or fat. In some embodiments, the FRSS further contains sodium citrate. In further some embodiments, the FRSS further contains citrus fiber. In some embodiments, at least or about 5% of the FRSS is the solid plant protein composition. In preferred embodiments, the solid plant protein composition FRSS enables a reduction in fat content in a food application (e.g., a baking application) utilizing the FRSS, when compared to the same food application utilizing an animal and/or dairy based shortening. In some embodiments, the reduction in fat is at least 10%, 20%, 30% or 40% when compared to the same food application utilizing an animal and/or dairy based shortening.

### K. Meat Analogues

In another embodiment, solid plant protein composition (e.g., mung bean protein) are included in a meat analogue. In some embodiments, the meat analogue contains one or more additional components selected from water, oil, disodium phosphate, optionally transglutaminase, starch and salt. In some embodiments, at least or about 10% of the meat analogue is the solid plant protein composition. In some embodiments, preparation of the meat analogue includes mixing the components of the meat analogue into an emulsion and pouring the emulsion into a casing that can be tied into a chubb. In some embodiments, chubs containing the meat analogue are incubated in a water bath at 50° C. for 2 hours. In further embodiments, the incubated chubbs are pressure-cooked. In some embodiments, the pressure-cooking occurs at 15 psi at about 121° C. for 30 minutes.

### L. Food Applications: Co-Ingredients

Various gums, phosphates, starches, preservatives, and other ingredients may be included in the food compositions containing the solid plant protein compositions.

Various gums useful for formulating one or more solid plant protein composition-based food products described herein include, e.g., konjac, gum acacia, Versawhip, Guar+Xanthan, Q-extract, CMC 6000 (Carboxymethylcellulose), Citri-Fi 200 (citrus fiber), Apple fiber, Fenugreek fiber.

Various phosphates useful for formulating one or more solid plant protein composition-based food products described herein include disodium phosphate (DSP), sodium hexamethaphosphate (SHMP), and tetrasodium pyrophosphate (TSPP). Although, the incorporation of these salts may be avoided as the solid pulse protein compositions described herein contain these salts which carry through to the downstream formulations.

Starch may be included as a food ingredient in the solid plant protein composition-based food products described herein. Starch has been shown to have useful emulsifying properties; starch and starch granules are known to stabilize emulsions. Starches are produced from plant compositions, such as, for example, arrowroot starch, cornstarch, tapioca starch, mung bean starch, potato starch, sweet potato starch, rice starch, sago starch, wheat starch.

In certain embodiments, the food composition contains an effective amount of an added preservative in combination with the solid pulse protein composition. The preservative may include ascorbic acid, citric acid, sodium benzoate, calcium propionate, sodium erythorbate, sodium nitrite, calcium sorbate, potassium sorbate, BHA, BHT, EDTA, tocopherols (Vitamin E) or antioxidants. M. Storage And Shelf Life of Food Compositions

In some embodiments, the food compositions containing the solid plant protein composition may be stable in storage at room temperature for up to 1, 2, 3, 4, 5, 6, 7, 8, 9, or 10 weeks. In some embodiments, the food compositions containing the solid plant protein composition may be stable for storage at room temperature for months, e.g., greater than 1, 2, 3, 4, 5, 6, 7, 8, 9, 10, 11, 12, or 13 months. In some embodiments, the food compositions containing the solid plant protein composition may be stable for refrigerated or freezer storage for months, e.g., greater than 1, 2, 3, 4, 5, 6, 7, 8, 9, 10, 11, 12, or 13 months. In some embodiments, the food compositions containing the solid plant protein composition may be stable for refrigerated or freezer storage for years, e.g., greater than 1, 2, 3, 4, 5, 6, 7, 8, 9, 10, 11, 12, or 13 years.

In some embodiments, storage as a dry material can increase the shelf life of the solid plant protein composition or a food composition containing the solid plant protein composition. In some embodiments, the solid plant protein composition or a food composition containing the solid plant protein composition is stored as a dry material for later reconstitution with a liquid, e.g., water. In some embodiments, the solid plant protein composition or the food composition is in solid (e.g., powdered) form, which may be less expensive to ship, lowers risk for spoilage and increases shelf-life (due to greatly reduced water content and water activity).

In various embodiments, a food composition (e.g., an egg-free liquid egg analog product) containing the solid plant protein composition has a viscosity of from about 300 CP to about 1500 cP (including all values in between) after storage at thirty days at 4° C. In some embodiments, the viscosity is less than about 1500, 1250, 1000, 900, 800, 700, 600, 575, 550, 525, 500, 475, 450, 425, 400, 375, 350, 325, or 300 cP (including all values in between 1500 and 300 cP) after storage for thirty days at 4° C., preferably after sixty days at 4° C. In some embodiments, the food composition has an initial viscosity of from about 300 to about 1500 cP and the viscosity of the food composition remains between about 300 and about 1500 cP over a period of thirty and/or sixty days at 4° C.

In some cases, the composition has a viscosity of less than 500 cP after storage for sixty days at 4° C. In various embodiments, a food composition (e.g., an egg-free liquid egg analog product) containing the solid pulse protein isolate composition has a viscosity of less than 450 cP after storage for thirty days at 4° C. In some cases, the composition has a viscosity of less than 450 cP after storage for sixty days at 4° C.

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Examples

The following paragraphs and examples are put forth so to provide those of ordinary skill in the art with a complete disclosure and description of how to make and use the methods and compositions of the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.
1. A solid plant protein composition comprising
   (a) one or more plant proteins;
   (b) one or more phosphate salts;
   (c) one or more Group I and/or Group II metal halide salts; and
   (d) optionally, one or more citrate salts.
2. The solid plant protein composition of paragraph 1, wherein the amount of the one or more plant proteins is at least about 60% by weight of the composition.
3. The solid plant protein composition of paragraph 1 or 2, wherein the one or more phosphate salts is selected from the group consisting of disodium phosphate (DSP), sodium hexamethaphosphate (SHMP), and tetrasodium pyrophosphate (TSPP).
4. The solid plant protein composition of any one of paragraphs 1-3, wherein the concentration of the one or more phosphate salts is an amount effective to decrease viscosity, modulate gelation, increase the denaturing temperature of the protein, increase the solubility of the plant protein composition, or combinations thereof.
4. The solid plant protein composition of paragraph 3, wherein the concentration of the one or more phosphate salts is from about 0.01% to about 5.0% by weight.
5. The solid plant protein composition of any one of paragraphs 1-4, wherein the Group I salt comprises sodium chloride, potassium chloride, and potassium iodide.
6. The solid plant protein composition of paragraph 5, wherein the Group I salt comprises sodium chloride.
7. The solid plant protein composition of any one of paragraphs 1-6, wherein the concentration of the one or more Group I/Group II salts is an amount effective to decrease viscosity, modulate gelation, increase the denaturing temperature of the protein, increase the solubility of the plant protein composition, or combinations thereof.
8. The solid plant protein composition of paragraph 3, wherein the concentration of the one or more Group I/Group II salts is from about 0.01% to about 5.0% by weight.
9. The solid plant protein composition of any one of paragraphs 1-8, wherein the composition comprises one or more citrate salts.
10. The solid plant protein composition of paragraph 9, wherein the one or more citrate salts is selected from the group consisting of tripotassium citrate (TCP) and trisodium citrate.
11. The solid plant protein composition of paragraph 10, wherein the concentration of the one or more citrate salts is an amount effective to decrease viscosity, modulate gelation, increase the denaturing temperature of the protein, increase the solubility of the plant protein composition, or combinations thereof.
12. The solid plant protein composition of paragraph 11, wherein the concentration of the one or more phosphate salts is from about 0.01% to about 5.0% by weight of the composition.
13. The solid plant protein composition of any one of paragraphs 1-12, wherein the particle size is selected from the group consisting of Dx (10) less than about 10 microns, Dx (50) less than about 30 microns, Dx (90) less than about 70 microns, and combinations thereof.
14. The solid plant protein composition of any one of paragraphs 1-13, wherein the protein is cross-linked.
15. The solid plant protein composition of paragraph 14, wherein the protein was crosslinked with one or more enzymes selected from the group consisting of transglutaminase, sortase, subtilisin, tyrosinase, laccase, peroxidase, or lysyl oxidase.
16. The solid plant protein composition of paragraph 15, wherein the crosslinking enzyme is transglutaminase.
17. The solid plant protein composition of any one of paragraphs 14-16, wherein the particle size is selected from the group consisting of Dx (10) less than about 1 micron, Dx (50) less than about 30 microns, Dx (90) less than about 70 microns, and combinations thereof.
18. The solid plant protein composition of any one of paragraphs 1-17, wherein the plant is a pulse.
19. The solid plant protein composition of paragraph 18, wherein the pulse is selected from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius.
20. The solid plant protein composition of paragraph 19, wherein the pulse is mung bean (Vigna radiata or V. radiata) and/or any varietal or cultivar of V. radiata.
21. A method for producing a solid plant protein composition comprising:
   (a) suspending a purified plant protein in a solvent;
   (b) adding one or more phosphate salts, one or more Group I and/or Group II metal halide salts, and optionally one or more citrate salts, to the suspended purified plant protein to form a plant protein composition; and
   (c) drying the plant protein composition to form a solid plant protein composition.
22. The method of paragraph 28, wherein the purified plant protein is suspended to a total solids from about 10% to about 20%.
23. The method of any one of paragraphs 21 or 22, wherein the one or more phosphate salts are selected from the group consisting of disodium phosphate (DSP), sodium hexamethaphosphate (SHMP), and tetrasodium pyrophosphate (TSPP).
24. The method of paragraph 23, wherein the concentration of the one or more phosphate salts is from about 0.01% to about 5.0% by weight of the composition.
25. The method of any one of paragraphs 21-24, wherein the Group I salt comprises sodium chloride, potassium chloride, and potassium iodide.
26. The method of paragraph 25, wherein the Group I salt comprises sodium chloride.
27. The method of paragraph 25 or 26, wherein the concentration of the one or more Group I/Group II metal halide salts is from about 0.01% to about 5.0% by weight of the composition.
28. The method of any one of paragraphs 21-27, wherein the composition comprises one or more citrate salts.
29. The method of paragraph 28, wherein the one or more citrate salts is selected from the group consisting of tripotassium citrate (TCP) and trisodium citrate.
30. The method of paragraph 28 or 29, wherein the concentration of the one or more phosphate salts is from about 0.01% to about 5.0% by weight of the composition.
31. The method of any one of paragraphs 21-30, further comprises adding transglutaminase enzyme to crosslink the protein.
32. The method of paragraph 36, wherein the transglutaminase is added to the slurry at a concentration of about 0.0001 to 0.5% (w/w).
33. The method of paragraph 32, wherein the concentration of transglutaminase is about 0.0001 to 0.1%.
34. The method of any one of paragraphs 31-33, wherein the mixture is incubated at about 50° C. from about 15 to about 90 minutes.
35. The method of paragraph 34, wherein the mixture is subsequently rapidly heated to 70° C. and heated for about 1 to about 5 minutes.
36. The method of paragraph 35, wherein the mixture is subsequently rapidly cooled to less than about 50° C. and centrifuged.
37. The method of any one of paragraphs 21-36, wherein the plant is a pulse.
38. The method of paragraph 37, wherein the pulse is selected from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius.
39. The method of paragraph 38, wherein the pulse is mung beans (Vigna radiata or V. radiata) and/or any varietal or cultivar of V. radiata.
40. A food product comprising the protein composition of any one of paragraphs 1-20.
41. An egg-substitute comprising the protein composition of any one of paragraphs 1-20.

### EXAMPLES

### Example 1. Production of 3A

Milled mung beans were solubilized in water at pH 7.0, followed by the addition of 0.3% w/w sodium chloride. The solubilized product was centrifuged to separate the light phase (1B), which included mung bean proteins, from the heavy phase (1A), which included unwanted starch, fiber, fat, and other proteins. The light phase (1B) was adjusted to pH 5.2, the approximate isoelectric point of the desired mung bean proteins, followed by another centrifugation step to separate the heavy phase (3A), where the desired mung bean protein was located, from the light phase (3B). The heavy phase (3A) was resuspended in water at 10-20% total solids, pH adjusted to 6.0-6.2, followed by pasteurization at 72° C. for 45 seconds before a final pass through the spray dryer to generate protein isolate.

### Example 2. Production of 3A+Salts

3A was adjusted to a 10-20% total solids. A mixture of tetrasodium pyrophosphate (TSPP), tripotassiumm citrate (TPC) and sodium chloride, NaCl (0.3% TSPP, 0.15% TPC, and 0.3% NaCl) was added to the 3A slurry. The slurry was incubated at 52° C. for fifteen (15) minutes followed by pasteurization at 72° C. for six (6) minutes. Visual observation showed a thin liquid, i.e., low viscosity protein solution.

### Example 3. Production of 3A+Salts+Transglutaminase (TG)

3A was adjusted to a 10-20% total solids. A mixture of TPC, TSPP, NaCl, and transglutaminase (0.15% TPC, 0.3% TSPP, 0.3% NaCl, and 0.03% TG) was added to the 3A slurry. The slurry was incubated at 52° C. for fifteen (15) minutes followed by pasteurization at 72° C. for six (6) minutes. Visual observation showed a thin liquid, i.e., low viscosity protein solution.

### Example 4. Quantifying the Extent of TG Crosslinking

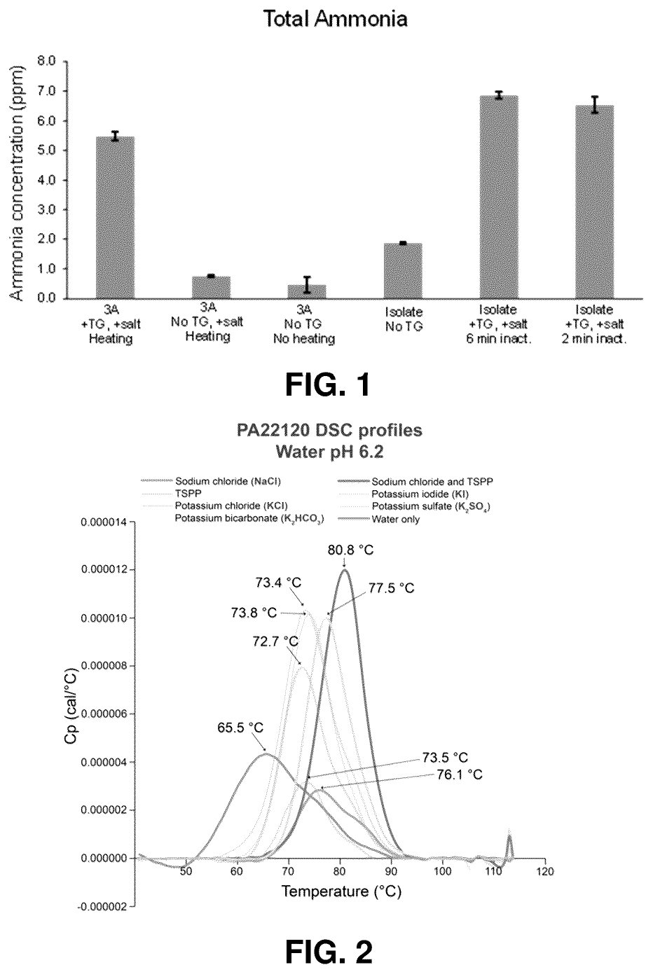

The amount of TG crosslinking was quantified using an ammonia quantification assay (see FIG. 1). During the reaction, as TG is crosslinking two protein molecules, an ammonium ion is released. The ammonia quantification assay uses a deproteinizer which denatures any protein and inactivates enzymes, including TG. The deproteinizer converts ammonium ions into ammonia. Three other reagents are then added and incubated at 37° C. while shielded from light which leads to the formation of a blue product. The absorption of UV light at a given wavelength is then measured and the amount of ammonia present in each sample is calculated based on a standard curve. Because ammonia can be present in the samples for reasons other than TG crosslinking (e.g., protein deamidation) a control sample with no TG was used to determine how much ammonia is generated as a direct result of the TG crosslinking.

For the experiments described above, the ammonia values detected were as expected. The 3A including TG and salt contained ˜5.5 ppm ammonia while the 3A without TG (with salt and heating) had ˜0.8 ppm meaning that the TG crosslinking generated ˜4.7 ppm. This is similar to the results using a protein isolate (control) which saw ˜6.7 ppm with TG and ˜1.9 ppm without TG giving ˜4.8 ppm ammonia generated from TG crosslinking. Overall, the similarity in the measured amounts of ammonia generated from TG suggests that the 3A crosslinked sample performed similarly to the control protein isolate sample.

### Example 5. Particle Size Distribution and Viscosity of 3A, 3A+Salts, and 3A+Salts+Tg

Samples containing either resuspended 3A or protein isolate were mixed with 0.15% TPC, 0.3% TSPP, and 0.3% NaCl and split into additional sample cups where one sample contained TG and one sample did not contain TG. All samples were incubated at 52° C. for 15 minutes then pasteurized at 72° C. for 6 minutes to deactivate the enzyme. Following preparation, the samples were analyzed for PSD using the Mastersizer (Malvern) and for viscosity using the viscometer (Brookfield). Looking at the percentile values for the 3A including salts [Table 1], the sample with TG shows a smaller particle size distribution where 10% of the particles are below 0.761 mm compared to the sample without TG showing 10% of the particles are below 5.96 mm. The protein isolate samples had similar PSD profiles where 10% of the particles are below 0.4-0.5 mm, which is similar to the 3A with TG sample.

Looking at the viscosities, the 3A and protein isolate samples containing TG had slightly higher viscosities, 39 and 42 cP, respectively, compared to the no TG containing samples, 30 and 33 cP, respectively, yet still within error of one another. Overall, the PSD measurements showed smaller particle sizes for the TG reaction, a result not observed with standard isolate extractions.

TABLE 1
Particle Size Distribution and Viscosity

| | Dx(10) | Dx(50) | Dx(90) | Viscosity |
|---|---|---|---|---|
| Sample | (μm) | (μm) | (μm) | (cP) |
| 3A with salt and no TG | 5.96 | 32.1 | 74.2 | 30 |
| 3A with salt and TG | 0.761 | 26.2 | 63.4 | 39 |
| Isolate with salt and no | 0.427 | 1.58 | 16.4 | 33 |
| TG | | | | |
| Isolate with salt and TG | 0.482 | 2.58 | 37.1 | 42 |

### Example 6. Thermal Stability of Purified Pulse Protein with Various Salts

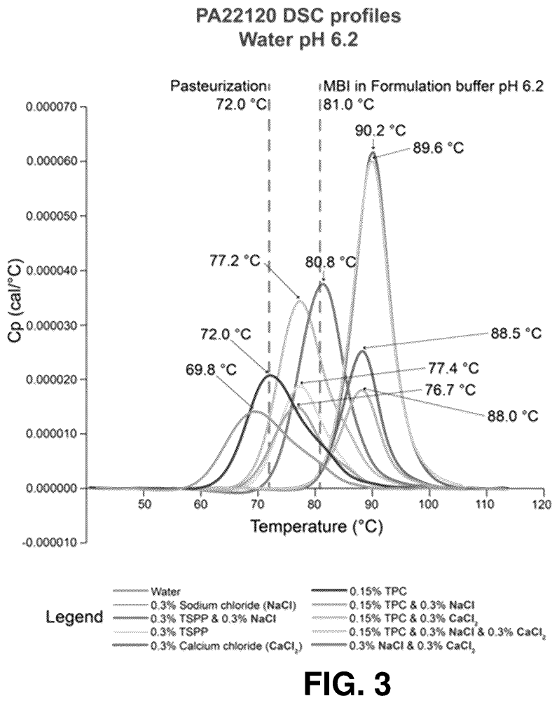

The thermal stability of a purified pulse protein (mung bean protein) in water alone, or with one or more salts, was evaluated using differential scanning calorimetry (DSC). The results are shown in FIGS. 2 and 3. As shown in FIG. 2, the melting point of the pulse protein (mung bean protein) in water is 65.5° C. Addition of sodium chloride increased the melting point to 76.1° C. but decreased the area under the curve compared to water suggesting poorer/lower solubility of the protein. The addition of various potassium salts increased the melting point compared to water (although not as much as sodium chloride, NaCl) but with larger areas under the curve (AUC). Addition of a phosphate salt (tetrasodium pyrophosphate, TSPP), alone or in combination with NaCl, exhibited the highest melting points, 77.5° C. and 80.8° C. FIG. 3 shows results with different phosphate salts, TSPP and tricalcium phosphate, TPC. Alone, TSPP exhibits a higher melting point than TPC. However, TPC in combination with CaCl₂) or CaCl₂) and NaCl shows the largest increase in the melting point with the combination of TPC, CaCl₂) and NaCl exhibiting the largest AUC.

### Example 7. Effect of Salts on Viscosity of Protein Composition Solutions

Pulse (mung bean) protein isolate, 3A, was resuspended in water in combination with TSPP, NaCl, and TCP. One aliquot further contains transglutaminase (TG), while another aliquout served as a control (no TG). Both aliquots were subjected to incubation conditions followed by deactivation conditions. Bother aliquots remained thin liquids. This is evidence that the presence of salts, phosphate salts in particular, decreases viscosity thereby mitigation gelation. This is important for both upstream and downstream applications.

### Example 8. Functionality and Sensory Evaluation of Various Protein Isolate

### Formulations

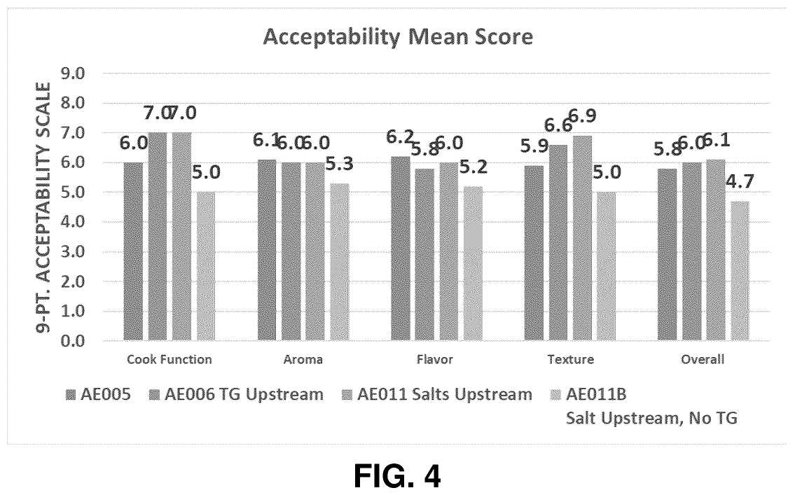

Functionality and sensory evaluation of various protein isolates formulated as a liquid egg product were conducted. All liquid egg products were formulated the same; only the nature of the protein isolate differed. Four (4) formulations were compared: AE005 (Control, TG and salts downstream); AE006 2 (TG and salts upstream); AE011 (Formulation 3) (salts (TSPP, TPC, and NaCl) upstream, TG downstream); and AE011 (Formulation 4) (salts upstream, no TG). The results are shown in FIG. 4 (from left to right: AE005 to AE011). Unexpectedly, AE011 (Formulation 3) performed as well (cook function and aroma) or better (flavor and texture) and better overall, than AE011 (Formulation 4). A summary of the data is shown in Table 2.

TABLE 2
Summary of characteristics of various formulations

| Isolate | Conditions | Functionality | Texture | Flavor | Viscosity | Conductivity | pH |
|---|---|---|---|---|---|---|---|
| AE005 | Control | 6 | 5.9 | 6.2 | 533 | 10.43 | 6.2 |
| hybrid | | | | | | | |
| AE006 | Salts and TG | 7 | 6.6 | 5.8 | 322 | 11.74 | 6.03 |
| hybrid | upstream | | | | | | |
| AE011 | Salts upstream | 7 | 6.9 | 6.5 | 203 | 12.08 | 6.18 |
| hybrid | TG | | | | | | |
| | downstream | | | | | | |
| AE011 | Salts upstream | 5 | 5.0 | 5.2 | 186 | 12.12 | 6.18 |
| hybrid | No TG | | | | | | |

AE011 hybrid (salts upstream, TG downstream) performed equally well as AE006 hybrid (salts and TG upstream) in functionality but performed better in texture and flavor. Moreover, AE011 had a lower viscosity than AE006.

### Example 9. Shelf-Life Dependent Viscosity and Functionality Studies

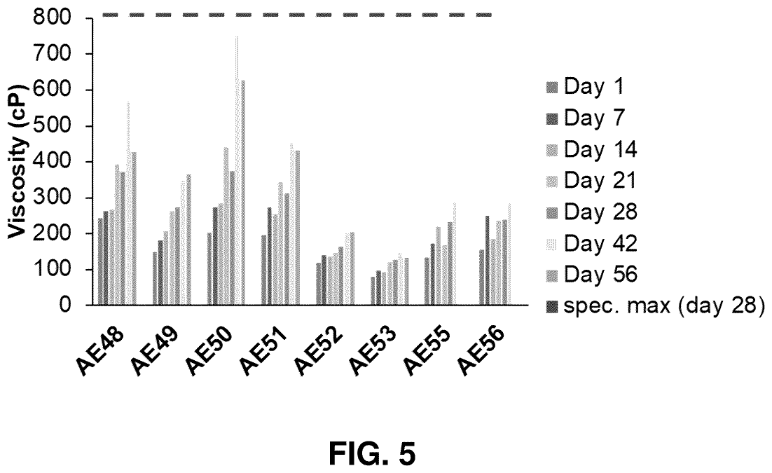

A series of compositions, AE048-AE-056, with different pilot conditions were evaluated for viscosity and functionality over a period of 56 days. A description of the different pilot conditions and the results are shown in Table 3. Several of the compositions showed little or no change in texture between days 1 and 56 and in some cases showed an improvement. All the compositions had a final viscosity of less than 650 cP and in some cases less than 500, 450, 300 or 250 cP. The viscosity of the various compositions at days 1, 7, 14, 21, 28, 42, and 56 are shown in FIG. 5. The results are in order from left to right for day 1 to day 56 (i.e., first bar is day 1, second bar is day 14, etc.).

TABLE 3
Functionality and Viscosity Data

| Isolate | | Texture | Texture | Final |
|---|---|---|---|---|
| # | Pilot Conditions | Day 1 | Day 56 | Viscosity |
| AE048 | Flow through tank | 6.6 | 6.5 | 428 |
| | control | | | |
| AE049 | 50% increase TG | 6.4 | 7 | 365 |
| AE050 | 75% increase TG | 6.2 | 7.5 | 628 |
| AE051 | 18 minutes incubation | 7.2 | 6.8 | 434 |
| AE052 | 15 minutes incubation | 6.8 | 6 | 204 |
| AE053 | 55° C. incubation | 7.3 | 6 | 134 |
| AE055 | 18 minutes incubation + | 6.8 | N/A | N/A |
| | 50% TG | | | |
| AE056 | 18 minutes incubation + | 6.7 | N/A | N/A |
| | 55° C. | | | |

What is claimed is:

1. A solid plant protein composition comprising
(a) one or more plant proteins;
(b) one or more phosphate salts;
(c) one or more Group I and/or Group II metal halide salts; and
(d) optionally, one or more citrate salts.

2. The solid plant protein composition of claim 1, wherein the amount of the one or more plant proteins is at least about 60% by weight of the composition.

3. The solid plant protein composition 1 comprising one or more phosphate salts, wherein the one or more phosphate salts is selected from the group consisting of disodium phosphate (DSP), sodium hexamethaphosphate (SHMP), and tetrasodium pyrophosphate (TSPP), optionally, wherein the concentration of the one or more phosphate salts is an amount effective to decrease viscosity, modulate gelation, increase the denaturing temperature of the protein, increase the solubility of the plant protein composition, or combinations thereof, and optionally, wherein the concentration of the one or more phosphate salts is from about 0.01% to about 5.0% by weight.

4. The solid plant protein composition of claim 1, wherein (a) the Group I salt comprises sodium chloride, potassium chloride, and potassium iodide; and/or (b) wherein the concentration of the one or more Group I/Group II salts is an amount effective to decrease viscosity, modulate gelation, increase the denaturing temperature of the protein, increase the solubility of the plant protein composition, or combinations thereof, optionally, wherein the concentration of the one or more Group I/Group II salts is from about 0.01% to about 5.0% by weight.

5. The solid plant protein composition of claim 1, wherein the composition comprises one or more citrate salts, optionally, wherein the concentration of the one or more citrate salts is an amount effective to decrease viscosity, modulate gelation, increase the denaturing temperature of the protein, increase the solubility of the plant protein composition, or combinations thereof; optionally, wherein the concentration of the one or more citrate salts is from about 0.01% to about 5.0% by weight of the composition.

6. The solid plant protein composition of claim 5, wherein the one or more citrate salts is selected from the group consisting of tripotassium citrate (TCP) and trisodium citrate.

7. The solid plant protein composition of claim 1, wherein the particle size is selected from the group consisting of Dx (10) less than about 10 microns, Dx (50) less than about 30 microns, Dx (90) less than about 70 microns, and combinations thereof.

8. The solid plant protein composition of claim 1, wherein the protein is cross-linked, optionally, with one or more enzymes selected from the group consisting of transglutaminase, sortase, subtilisin, tyrosinase, laccase, peroxidase, or lysyl oxidase.

9. The solid plant protein composition of claim 7, wherein the particle size is selected from the group consisting of Dx (10) less than about 1 micron, Dx (50) less than about 30 microns, Dx (90) less than about 70 microns, and combinations thereof.

10. The solid plant protein composition of claim 1, wherein the plant is a pulse, selected from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius.

11. The solid plant protein composition of claim 10, wherein the pulse is mung bean (Vigna radiata or V. radiata) and/or any varietal or cultivar of V. radiata.

12. A method for producing a solid plant protein composition comprising:
(a) suspending a purified plant protein in a solvent;
(b) adding one or more phosphate salts, one or more Group I and/or Group II metal halide salts, and optionally one or more citrate salts, to the suspended purified plant protein to form a plant protein composition; and
(c) drying the plant protein composition to form a solid plant protein composition.

13. The method of claim 12, wherein the purified plant protein is suspended to a total solids from about 10% to about 20%.

14. The method of claim 12 wherein: (a) the Group I and/or Group II metals comprises one or more phosphate salts, wherein the one or more phosphate salts are selected from the group consisting of disodium phosphate (DSP), sodium hexamethaphosphate (SHMP), and tetrasodium pyrophosphate (TSPP), optionally, wherein the concentration of the one or more phosphate salts is from about 0.01% to about 5.0% by weight of the composition; (b) the Group I salt comprises sodium chloride, potassium chloride, and potassium iodide, optionally, wherein the concentration of the one or more Group I/Group II metal halide salts is from about 0.01% to about 5.0% by weight of the composition and/or (c) wherein the composition comprises one or more citrate salts selected from the group consisting of tripotassium citrate (TCP) and trisodium citrate, optionally, wherein the concentration of the one or more phosphate salts is from about 0.01% to about 5.0% by weight of the composition.

15. The method of claim 12, further comprises adding transglutaminase enzyme to crosslink the protein, optionally, wherein: (a) the transglutaminase is added to the slurry at a concentration of about 0.0001 to 0.5% (w/w); or (b) the concentration of transglutaminase is about 0.0001 to 0.1%.

16. The method of claim 12, wherein: (a) the mixture is incubated at about 50° C. from about 15 to about 90 minutes; (b) the mixture is subsequently rapidly heated to 70° C. and heated for about 1 to about 5 minutes; and/or (c) the mixture is subsequently rapidly cooled to less than about 50° C. and centrifuged.

17. The method of claim 12, wherein the plant is a pulse, selected from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius.

18. The method of claim 17, wherein the pulse is mung beans (Vigna radiata or V. radiata) and/or any varietal or cultivar of V. radiata.

19. A food product comprising the protein composition of claim 1, optionally wherein the food product is an egg-substitute.
